(19) United States
(12) Patent Application Publication
(10) Pub. No.: US 2014/0161673 A1
(43) Pub. Date: Jun. 12, 2014

(54) AIR FRESHENING SYSTEM AND METHOD

(71) Applicant: Scott David Hammer, Dallas, TX (US)

(72) Inventor: Scott David Hammer, Dallas, TX (US)

(21) Appl. No.: 13/902,028

(22) Filed: May 24, 2013

Related U.S. Application Data
(63) Continuation-in-part of application No. 13/843,232, filed on Mar. 15, 2013.
(60) Provisional application No. 61/728,603, filed on Nov. 20, 2012.

(51) Int. Cl. A61L 9/12; F24F 13/08

(52) U.S. Cl. CPC A61L 9/12; F24F 13/082
USPC 422/124; 29/525.01

(57) ABSTRACT

The present invention supports an airflow register accessory comprising an accessory having an outer and an inner surface and defining vents, a fastening mechanism located on the inner surface of the accessory, and a scented insert component located on the inner surface adjacent and accessorizing the vents. In a preferred embodiment, the accessory may be easily attached to and detached from the room-facing surface of an airflow register. Upon air flow from an HVAC system through the airflow register and airflow register accessory, a pleasant scent may be imparted to the room via release of scent from the scented component.

### RELATED APPLICATION DATA

This application is a continuation-in-part of U.S. application Ser. No. 13/843,232, filed on Mar. 15, 2013, which claims the benefit of U.S. Provisional Application No. 61/728,603, filed on Nov. 20, 2012.

### TECHNICAL FIELD

The present invention relates in general to a system and method for providing an accessory for an air conditioning or heating air flow register such as are installed in the ceiling, walls or floors of homes and/or other buildings.

### BACKGROUND OF THE INVENTION

Air flow registers allow the flow of heated or cooled air into rooms of homes, offices and other buildings. Generally, such registers are made of metal and have various vents through which air flows. They may have a lever which can regulate the amount of air which flows through a given register.

Various devices have been used in the past to scent the air of a given room or area in a building. However, such devices may require electricity or heat in order to volatilize the scent so that it distributes throughout the area of interest. The problem with prior art systems is the inability to conceivably, effectively, and easily access the airflow passing through the airflow register to scent the air in the room.

### SUMMARY OF THE INVENTION

The present invention supports an airflow register accessory comprising an accessory having an outer and an inner surface and defining vents, a fastening mechanism located on the inner surface of the accessory, and a scented component located on the inner surface adjacent and accessorizing the vents. In a preferred embodiment, the accessory may be easily attached to and detached from the room-facing surface of an airflow register. Upon air flow from an HVAC system through the airflow register and airflow register accessory, a pleasant scent may be imparted to the room via release of scent from the scented component.

### BRIEF DESCRIPTION OF THE DRAWINGS

FIG. 1 is a front perspective environmental view illustrating an embodiment of an airflow register accessory in accordance with the principles of the present invention installed on an airflow register (the airflow register which is not part of this invention is shown in phantom throughout the drawings).

FIG. 2 is an exploded perspective environmental view illustrating an embodiment of an airflow register accessory in accordance with the principles of the present invention in relation to an airflow register, shown in phantom, on which it is installed in use.

FIG. 3 is a back view illustrating the airflow register accessory installed on an airflow register, shown in phantom.

FIG. 4 is a back exploded perspective view of an embodiment of the airflow register accessory in accordance with the principles of the present invention in relation to an airflow register, shown in phantom, on which it is installed in use.

FIG. 5 is a side view of an embodiment of the airflow register accessory as installed on an airflow register (in phantom) with the arrows illustrating air flow direction through the installed airflow register accessory in use.

FIG. 6 is a section view along the 6-6 section line of FIG. 3 of an embodiment of the airflow register accessory as installed on an airflow register (in phantom) with the arrows illustrating air flow direction through the installed airflow register accessory in use.

FIG. 7 is a front view of an embodiment of the airflow register accessory illustrating an optional perforated slot portion.

FIG. 8 is a rear perspective view illustrating another embodiment of an airflow register accessory in accordance with the principles of the present invention.

FIG. 9 is a section view along the 9-9 section line of FIG. 8 of the embodiment of the airflow register accessory as installed on an airflow register (in phantom) with the arrows illustrating air flow direction through the installed airflow register accessory in use.

FIG. 10 is a partial exploded perspective environmental view illustrating an embodiment of an airflow register accessory in accordance with the principles of the present invention in relation to an airflow register of differing size, shown in phantom, on which it is installed in use.

FIG. 11 is a rear perspective view illustrating another embodiment of an airflow register accessory in accordance with the principles of the present invention.

FIG. 12 is a section view along the 12-12 section line of FIG. 11 of the embodiment of the airflow register accessory as installed on an airflow register (in phantom) with the arrows illustrating air flow direction through the installed airflow register accessory in use.

FIG. 13 illustrates an accessory aide in accordance with the principles of the present invention.

FIG. 14 is an illustrative environmental view of a user applying the invention to an airflow register.

FIG. 15 is a rear perspective view illustrating another embodiment of an airflow register accessory adapted to receive a scented insert.

FIG. 16 is a rear perspective view illustrating an embodiment of a scented insert.

FIG. 16A is a front view of the scented insert embodiment of FIG. 16 illustrating the accessory facing surface.

FIG. 17 is a rear perspective view illustrating an embodiment of a scented insert.

FIG. 17A is a front view of the scented insert embodiment of FIG. 17 illustrating the accessory facing surface.

FIG. 18 is a rear perspective view illustrating an embodiment of a scented insert.

FIG. 18A is a front view of the scented insert embodiment of FIG. 18 illustrating the accessory facing surface.

FIG. 19 is a rear perspective view illustrating another embodiment of an airflow register accessory adapted to receive a scented insert.

FIG. 19A is a rear perspective view illustrating an embodiment of a scented insert.

FIG. 19B is a front view of the airflow register accessory embodiment of FIG. 19 illustrating the outer surface.

FIG. 19C is a front view of the scented insert embodiment of FIG. 19A illustrating the accessory facing surface.

FIG. 20 is a perspective view illustrating an alternate embodiment of the area surrounding an insert slot.

FIG. 20A is a zoomed in perspective view of FIG. 20 of the area surrounding insert slot showing a slot cover in an open position.

FIG. 21 is a perspective view illustrating another alternate embodiment of the area surrounding an insert slot.

FIG. 21A is a zoomed in perspective view of FIG. 21 of the area surrounding insert slot showing a slot cover in an open position.

FIG. 22 is a perspective view illustrating an embodiment of a scented insert.

### DETAILED DESCRIPTION

A system providing an airflow register accessory, an accessory aide and method of applying said accessory to an airflow register is herein disclosed.

The airflow register accessory comprises an accessory, a scented fabric, and a fastening mechanism for attaching the accessory to the room-facing side of an airflow register.

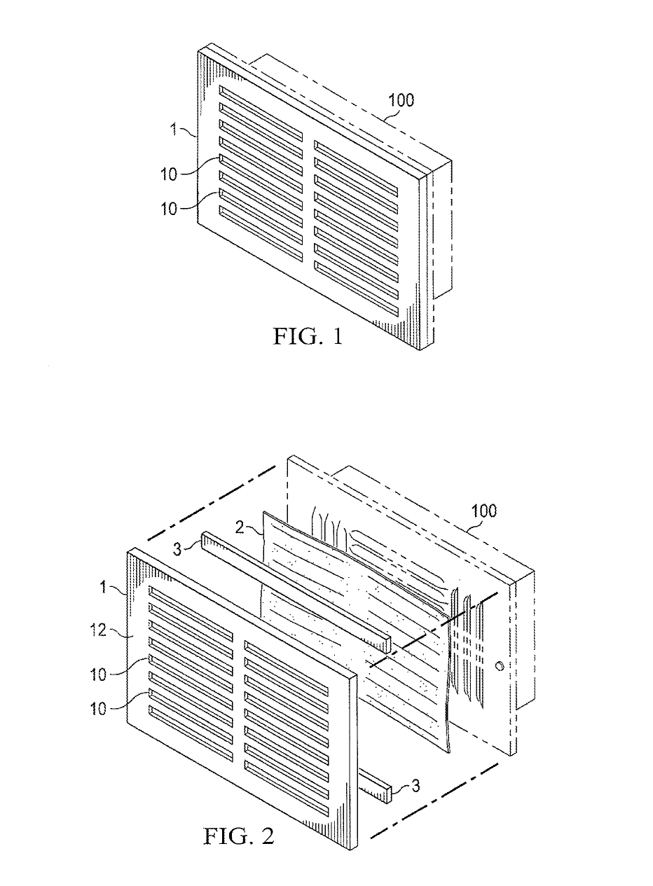

As best seen in FIGS. 1 and 2, accessory (1) of the airflow register accessory defines a plurality of vents (10) for air to flow through from the heating, ventilation and air conditioning unit (“HVAC”) system of a building, through an airflow register attached to a wall, ceiling or floor of the building, and then though the airflow register accessory of the invention when it is secured to said airflow register (100). FIG. 1 shows the airflow register accessory (1) installed on top over or covering the airflow register (100). Accessory is shown as rectangular, but could be square, circular, or other shapes, which depend on the shape of the air register. A user can also use a larger accessory (example shown in FIG. 10) and move it around as fragrance is dissipated.

FIG. 2 best illustrates the components of the airflow register accessory. The accessory (1) has an outer side (12) and an inner side (13) (not shown in FIG. 2). The airflow register accessory (1) further comprises a fastening mechanism (3) secured to the inner side of the accessory. The fastening mechanism (3) is attached to the inner side of the airflow register accessory (1), and in turn may be used to secure the airflow register accessory to an airflow register (100). Preferably the fastening mechanism (3) is secured to the top and bottom of the inner side (13) to avoid interference with screws, vent levers, and the like of a typical airflow register (100), but may be secured anywhere along the inner side (13) as to allow it to temporarily secure to any type of airflow register.

If the airflow register of interest is metal to which a magnet can adhere, the fastening mechanism can be one or more magnets. This allows for quick and easy attachment or detachment of the airflow register accessory (1) from the airflow register (100) of the room. Magnetic tape strips may also be used. If the accessory is made from non-magnetic material, it is convenient to use a magnetic tape strip that has an adhesive side for adhering to the inner side of the accessory.

Alternatively, other fastening mechanism such as hook and loop fasteners may be employed (not illustrated). In such case, the complement of the hook or the loop is attached to the airflow register (100) and the accessory secured thereto via the complementary fastener (not shown). Double-sided adhesive may also be used to attach the accessory to the airflow register. Less preferred, but possible, are fastening mechanism such as screws or bolts as they can secure the accessory to the airflow register, but make it more arduous to remove and replace the air accessory. If such are used, preferably they will extend through both the airflow register accessory and the airflow register and secure both to the wall, ceiling, or floor.

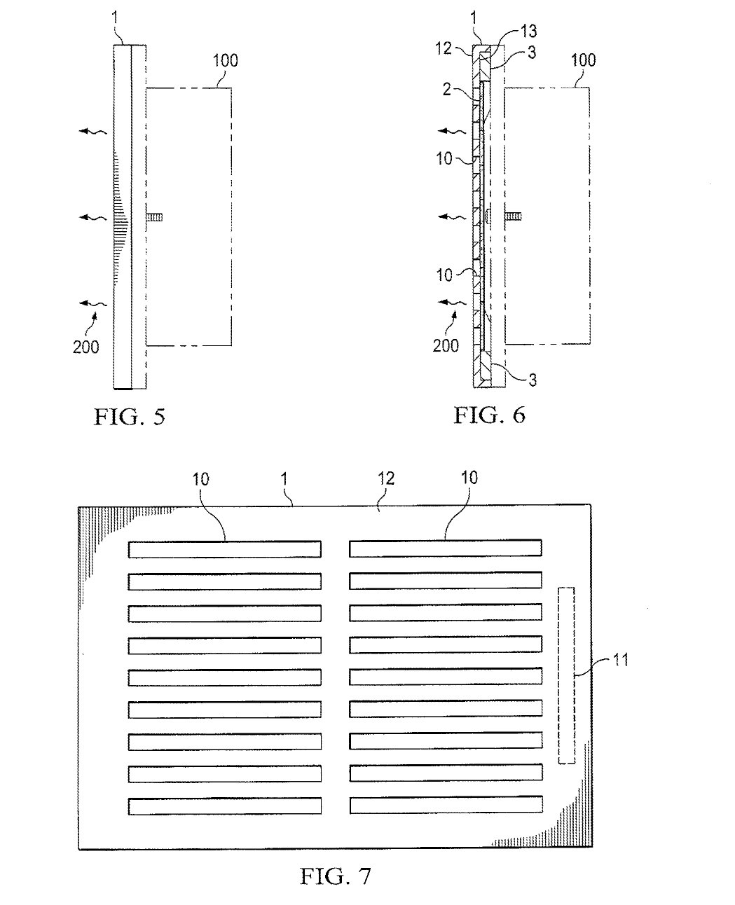

The accessory (1) further comprises a scenting material. FIG. 2 illustrates a scented fabric (2) used for the scenting material. The scented fabric (2) is attached to the inner side (13) of the accessory (1) adjacent the vent openings (10). The scented fabric (2) may be perforated or otherwise woven or spun so that it allows sufficient airflow there through (as illustrated in FIGS. 5 and 6). Alternatively, one or more slits which are oriented in the same way as the vents may be made in the scented fabric (2). The scented fabric (2) may be essentially rectangular to correspond to a rectangular vent area of the accessory, and have four edges—two long and two short. Preferably, at least the short edges are secured by adhesion or other wise to the inside of the accessory (1) so that the scented fabric (2) covers the vent openings (10) of the accessory (1). Airflow register accessory and/or scented fabric may also be other shapes to correspond to size and shape of the airflow register.

Preferably, the scented fabric comprises a substrate which may be made of natural fibers, synthetic fibers, or a mixture of natural fiber and synthetic fibers. Exemplary natural fibers that can be used to form the substrate include wood fibers and non-wood natural fibers such as vegetable fibers, cotton, straw, canes, silk, animal fiber, grasses, hemp, and corn stalks. Nonwoven fabrics are broadly defined as sheet or web structures bonded together by entangling fiber or filaments (and by perforating films) mechanically, thermally or chemically. They are typically flat, porous sheets that are made directly from separate fibers or from molten plastic or plastic film. Some examples of nonwoven materials are staple nonwovens, spun laid nonwovens, and air-laid paper. Woven fabric is a cloth formed by weaving. Synthetic fibers may include a variety of substrates known in the art. An exemplary fiber is polylactide fiber or PLA. PLA is a biodegradable thermoplastic derived from lactic acid.

The fabric material has a weight of approximately 0.2 to 2.0 oz/ft², but can have weights that vary from 0.05 to 4.55 oz/ft². An exemplary combination substrate for the fabric comprises a mixture of 0.1 wt. % to 1.00 wt. % of the polylactide fiber and about 10 wt. % to about 1.00 wt. % of the natural fiber. The fabric preferably is flame retardant, and can be composed of a polyester or natural material (woven or non-woven). The airflow rate should be extremely high on the order a thin mesh that freely allows airflow. The fabric component has a high degree of air permeability on the order of not impeding airflow by more than 5% to 10% by uncovered air flow rates.

The scented fabric component is preferably thin, but must be sturdy enough to withstand typical ventilation flows of HVAC systems without being torn or compromised such that it cannot perform the intended function. The ventilation rate is normally expressed by the volumetric flow rate of outside air being introduced to the HVAC. The typical units used are cubic feet per minute (CFM) or liters per second (L/s). The ventilation rate can also be expressed on a per person or per unit floor area basis, such as CFM/p or CFM/ft².

For residential housing, which mostly relies on air infiltration for ventilation, the common ventilation rate measure is the number of times the whole interior volume of air is replaced per hour, and is called air changes per hour (I or ACH; units of 1/h). During the winter, ACH may range from 0.50 to 0.41 in a tightly insulated house to 1.11 to 1.47 in a loosely insulated house.

The scenting fabric (2) has an air freshening agent loaded thereon. The air freshening agent must be able to adhere to the fabric but at least a portion thereof should be released when air flows through the scented fabric component. In the natural operation of the HVAC system, heated or cooled air will flow through the scenting material. Thus the air freshening agent may be chosen to release when air of any temperature flows through the scenting material at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting fabric so that the air freshening accessory can provide air freshening benefits for a pre-determined period of time. After the pre-determined period of time, it is contemplated that the airflow register accessory will be replaced. Alternatively, but less preferred, only the scented fabric component may be replaced.

A scented strip may be used instead of the scented fabric (2). The scented strip would comprise an adhesive surface that is secured to the inner surface (13) of accessory (1). The scented strip would also comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors.

The embodiment of FIG. 2 may also comprise an air filter. The air filter preferably would lie adjacent the surface of the scented fabric (2) opposite the inner surface (13) of accessory (1). The air filter may also lie between the inner surface (13) and scented fabric (2). The air filter would preferably be secured to the accessory (1) by the same method used to secure the scented fabric (2), but other methods may be used that maintain the air filter in place. Additionally, the scented fabric (2) and air filter may be fabricated into a single combined piece. If fabricated into a single combined piece, the single combined piece would be secured to the accessory (1) by the means stated above regarding the scented fabric (2). Any type of air filter may be used as long as it fits inside the accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

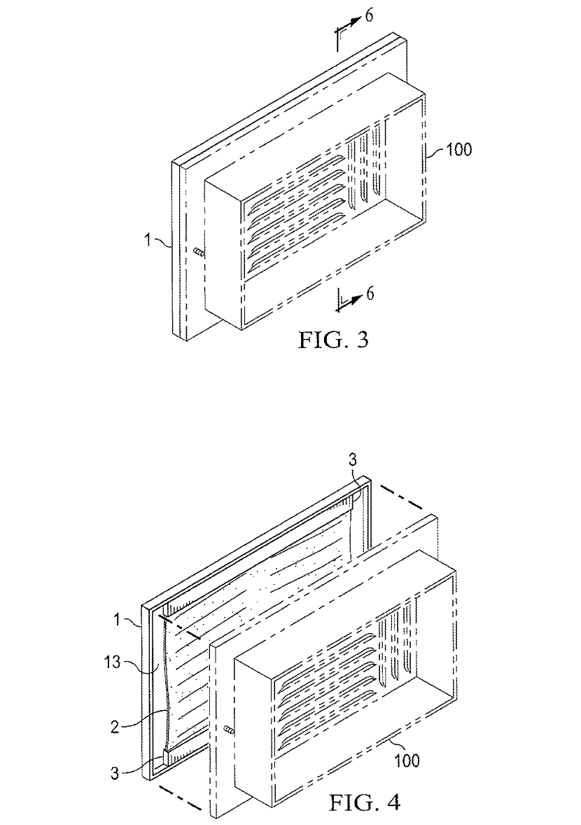

FIG. 3 illustrates a rear perspective view of a typical airflow register (100) with the accessory (1) secured flush with the airflow register (100). FIG. 3 shows the airflow register accessory (1) installed on top over or covering the airflow register. Accessory is shown as rectangular, but could be square, circular, or other shapes, which depend on the shape of the air register. A user can also use a larger accessory (example shown in FIG. 10) and move it around as fragrance is dissipated.

FIG. 4 also illustrates the components of the airflow register accessory of FIG. 2. The accessory (1) has an outer side (not shown in FIG. 4) and an inner side (13). The airflow register accessory (1) further comprises a fastening mechanism (3) secured to the inner side of the accessory. The fastening mechanism (3) is attached to the inner side (13) of the airflow register accessory (1), and in turn may be used to secure the airflow register accessory to an airflow register (100). Preferably the fastening mechanism (3) is secured to the top and bottom of the inner side (13) to avoid interference with screws, vent levers, and the like of a typical airflow register (100), but may be secured anywhere along the inner side (13) as to allow it to temporarily secure to any type of airflow register.

If the airflow register of interest is metal to which a magnet can adhere, the fastening mechanism can be one or more magnets. This allows for quick and easy attachment or detachment of the airflow register accessory (1) from the airflow register (100) of the room. Magnetic tape strips may also be used. If the accessory is made from non-magnetic material, it is convenient to use a magnetic tape strip that has an adhesive side for adhering to the inner side of the accessory.

Other fastening mechanisms such as hook and loop fasteners may be employed (not illustrated). In such case, the complement of the hook or the loop is attached to the airflow register (100) and the accessory secured thereto via the complementary fastener (not shown). Double-sided adhesive may also be used to attach the accessory to the airflow register. Less preferred, but possible, are fastening mechanism such as screws or bolts as they can secure the accessory to the airflow register, but make it more arduous to remove and replace the air accessory. If such are used, preferably they will extend through both the airflow register accessory and the airflow register and secure both to the wall, ceiling, or floor.

Alternatively, accessory (1) may comprise attachment extensions that extend inward from the inner surface (13). Fastening mechanisms (3) would be secured to the attachment mechanisms. By providing the attachment extensions, the thickness of the accessory may be varied and allow the same fastening mechanisms to be used. The extensions may also be cost effective as it allows for thinner fastening mechanisms to be used.

The accessory (1) further comprises a scenting material. FIG. 4 illustrates a scented fabric (2) used for the scenting material. The scented fabric (2) is attached to the inner side (13) of the accessory (1) adjacent the vent openings (10). The scented fabric (2) may be perforated or otherwise woven or spun so that it allows sufficient airflow therethrough (as illustrated in FIGS. 5 and 6). Alternatively, one or more slits which are oriented in the same way as the vents may be made in the scented fabric (2).

The scented fabric (2) may be essentially rectangular to correspond to a rectangular vent area of the accessory, and have four edges—two long and two short. Preferably, at least the short edges are secured by adhesion or other wise to the inside of the accessory (1) so that the scented fabric (2) covers the vent openings (10) of the accessory (1). Airflow register accessory and/or scented fabric may also be other shapes to correspond to size and shape of the airflow register.

The scenting fabric (2) has an air freshening agent loaded thereon, which is a scented oil or additive. The scented additive can include an essential oil or concentrated additive containing a concentrated fragrance or scent. When disposed on the fabric the scented additive is diluted with a water-based diluent up to 80% to 90% (or over) per weight of the scented additive. The diluent evaporates after application leaving the scented additive on the scented fabric (2) of the airflow register. The air freshening agent must be able to adhere to the fabric but at least a portion thereof should be released when air flows through the scented fabric component. In the natural operation of the HVAC system, heated or cooled air will flow through the scenting material. Thus the air freshening agent may be chosen to release when air of any temperature flows through the scenting material at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting fabric so that the air freshening accessory can provide air freshening benefits for a pre-determined period of time. After the pre-determined period of time, it is contemplated that the airflow register accessory will be replaced. Alternatively, but less preferred, only the scented fabric component may be replaced.

A scented strip may be used instead of the scented fabric (2). The scented strip would comprise an adhesive surface that is secured to the inner surface (13) of accessory (1). The scented strip would also comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors.

The embodiment of FIG. 4 may also comprise an air filter. The air filter preferably would lie adjacent the surface of the scented fabric (2) opposite the inner surface (13) of accessory (1). The air filter may also lie between the inner surface (13) and scented fabric (2). The air filter would preferably be secured to the accessory (1) by the same method used to secure the scented fabric (2), but other methods may be used that maintain the air filter in place. Additionally, the scented fabric (2) and air filter may be fabricated into a single combined piece. If fabricated into a single combined piece, the single combined piece would be secured to the accessory (1) by the means stated above regarding the scented fabric (2). Any type of air filter may be used as long as it fits inside the accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

FIG. 5 best illustrates the side view of the airflow register accessory attached to an airflow register in use. FIG. 5 shows the airflow register accessory (1) installed on top over or covering the airflow register (100). Preferably the accessory back lays flush with the airflow register's front surface. This allows the accessory to appear as part of the register and more natural.

FIG. 6 is a cross-section taken along the section line 6-6 of FIG. 3 where the airflow register accessory (1) is installed on top over or covering the airflow register (100). In a preferred embodiment, the airflow register accessory (1) adheres to the airflow register via magnetic attraction. The room constantly has a pleasant scent for a period of time from the HVAC system causing airflow (200) through the airflow register (100) and through the airflow register accessory (1) adhered thereto. When the scent diminishes or no longer is evident, the airflow register accessory may be replaced as a unit. Accessory (1) of the airflow register accessory defines a plurality of vents (10) for air to flow through from the heating, ventilation and air conditioning unit (“HVAC”) system of a building, through an airflow register attached to a wall, ceiling or floor of the building, and then though the airflow register accessory of the invention when it is secured to said airflow register (100). Accessory (1) is shown as rectangular, but could be square, circular, or other shapes, which depend on the shape of the air register. A user can also use a larger accessory (example shown in FIG. 10) and move it around as fragrance is dissipated.

As illustrated in FIG. 7, accessory (1) may further define a perforated slot portion (11). The perforated slot portion (11) may optionally be removed from the airflow register accessory; thereby creating a slot through which an airflow register's regulating lever may pass through, if the airflow register has such a lever. The perforated slot portion (11) may be punched out by hand pressure or by using an implement to push it out, such as a common screwdriver.

FIG. 8 illustrates an alternate embodiment for the airflow register accessory. This embodiment of the accessory (1) comprises a spacing wall (15). The spacing wall (15) is a thin strip extending inward perpendicularly from the accessory side wall and extending parallel to the accessory front wall a distance from the inner side (13).

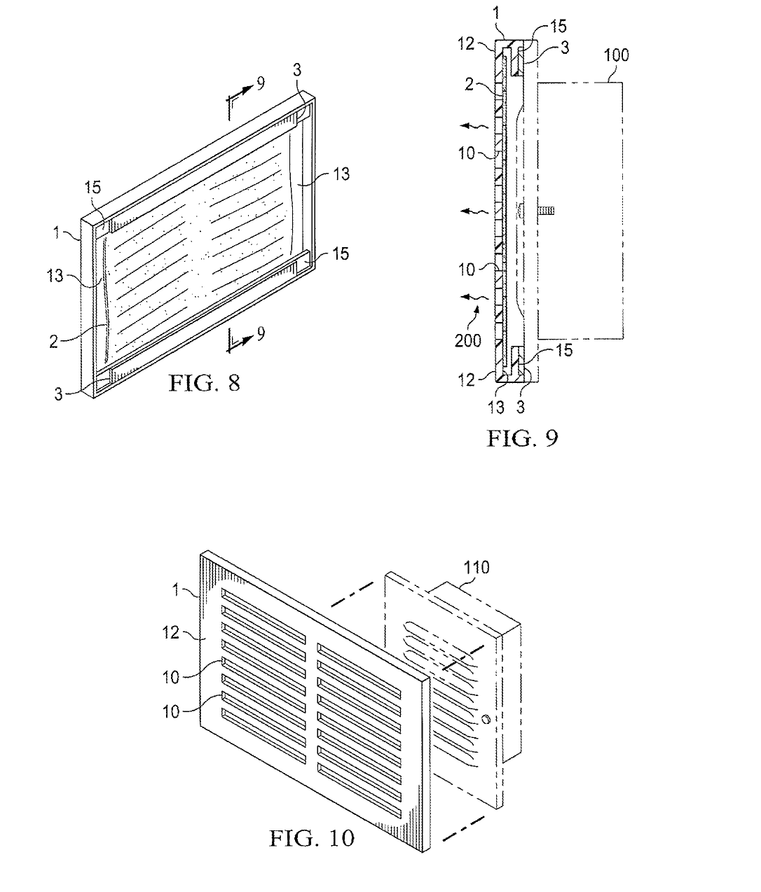

FIG. 9 is a section view along section line 9-9 of FIG. 8 that further illustrates the alternate embodiment of FIG. 8. The spacing wall (15) is preferably located a slight distance away from the accessory back edge as to allow clearance for the securing mechanism (3) to attach thereto and retain the flush securement of the accessory to the airflow register as sown in FIG. 5. The spacing wall (15) also provides more space between the spacing wall (15) and inner side (13) to secure the scenting material (2) to the accessory (1). Another advantage to the spacing wall (15) is that it helps reduce the width of the securing mechanism (3) and may reduce the weight of the accessory (1) to provide easier handling of the accessory (1).

As airflow registers may vary in size and shape, the airflow register accessory (1) may also be created in various sizes. For example, as illustrated in FIG. 10, a user may use a larger accessory with a smaller airflow register (110). This configuration may allow the accessory to last longer as less of the scenting material area is used, allowing the user to later adjust the accessory to use the remaining scenting material.

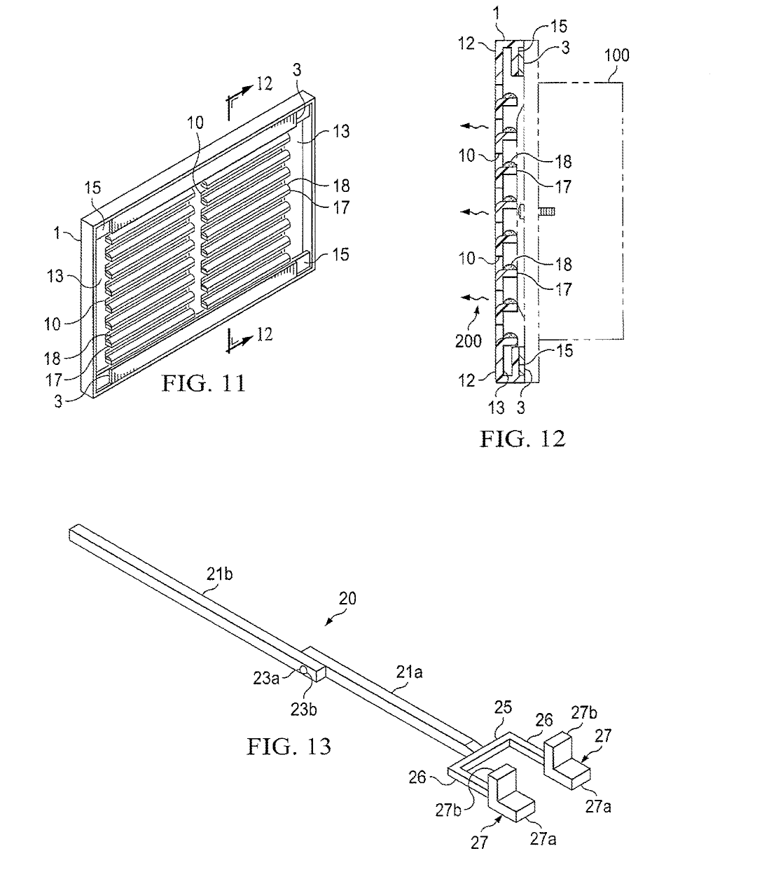

FIG. 11 illustrates another alternate embodiment of the airflow register accessory. The embodiment is similar to the embodiment described above in FIG. 4 with an alteration to the scenting material. In lieu of a scented fabric, the embodiment of FIG. 11 employs a scented gel (18). The inner side (13) of accessory (1) comprises multiple inner ledges (17) that extend out from the inner side surface. The inner ledges (17) each have four edges, two long and two short. One of the long edges is secured to or extends from a long edge of vent opening (10) providing the inner ledge (17) with a surface adjacent the vent opening (10) and a surface adjacent the inner side (13). On the surface adjacent the vent opening (10), the inner ledge (17) comprises a scented gel (18) secured thereto which acts as the air freshening agent.

A scented strip may be used instead of the scented gel (18). The scented strip would comprise an adhesive surface that is secured to the inner surface (13) of accessory (1) or secured to the inner ledge (17) on the surface adjacent the vent opening (10). The scented strip would also comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors.

The embodiment of FIG. 11 may also comprise an air filter. The air filter would preferably lie adjacent the long non-secured edges of inner ledges (17). The air filter would preferably be secured to the accessory (1) by tape, glue or adhesive, but other methods may be used that maintain the air filter in place. Any type of air filter may be used as long as it fits inside the accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

FIG. 12 further illustrates the embodiment shown in FIG. 11. FIG. 12 shows inner ledges (17) and scented gel (18) residing in front of the airflow register (100) when airflow register accessory (1) is attached to the airflow register (100). The airflow (200) flows out of the airflow register (100) passes along the scented gel (18) residing on the inner ledges (17), through vent openings (10) and out to the room.

The scented gel is preferably a polymeric, polyester, gelatin, polysaccharide gel or a glycol base element ranging in 50% to 70% by weight of the gel composition. The properties of the gel range from free-flowing liquids to liquid gels and self-supporting solid gels with a wide range of textures, setting temperatures and melting temperatures. Preferably the gel is self supporting gel so that it remains in the location it is applied on the device despite movement or rotation of the device. The gel composition is dilute when applied, but firms significantly after application to the airflow register. The gel when applied to the airflow register has a diluent of water in the range of 30% to 50% by weight of the gel composition, and includes anti-microbial additives to prevent microbial contamination and growth in the range of 0.20% to 5% of weight of the gel composition.

The gel composition has a scented fragrance component in the overall range of 0.75% to 3.75% by weight of the gel composition, and a colorant of 0.05% to 2.75% by weight of the gel composition.

The gel composition may also be composed of a viscoelastic gel having carrageenan and selected polymers, each polymer having either a cationic nitrogen content of at least about 3 wt. % and a weight average molecular weight of less than about 800,000 Dalton, and is selected from the group consisting of poly(diallyldimethyl ammonium halide), poly(DADMAC), and combinations thereof, or a cationic nitrogen content of less than about 3 wt. % and a weight average molecular weight of greater than about 1,000,000 Dalton. The polymer may be selected from cationic guar gum, a cationic cellulose, a cationic starch, hydrophobically-modified versions thereof, and combinations thereof.

These properties allow the gel to continuously release a scented aroma over an extended period of time. As with the other embodiments, it is contemplated that the airflow register accessory will be replaced once the air freshening agent has dissipated.

In addition to the airflow register accessory (1), the system of the invention comprises an accessory aide (20) as shown in FIG. 13. The accessory aide (20) comprises an upper portion (21a) and a lower portion (21b). The upper portion (21a) further comprises a holding section (25) at one of its ends. At the opposite end the holding section (25) on upper portion (21a) extends an attaching extension (23a) perpendicular to the upper portion (21a). Lower portion (21b) defines an attaching aperture (23b) at one of its ends. The attaching aperture (23b) is complimentary to the attaching extension (23a). The attaching extension (23a) and attaching aperture (23b) may also be located in different locations that allow attaching extension (23a) and attaching aperture (23b) to interact in a similar manner which is to connect the upper portion (21a) and lower portion (21b). Preferably upper and lower portions (21a and 21b) are removably attached or pivot where adjoined to allow for easier storage. Upper and lower portions (21a and 21b) may also be part of a singular piece where attachment of the two sections in not necessary.

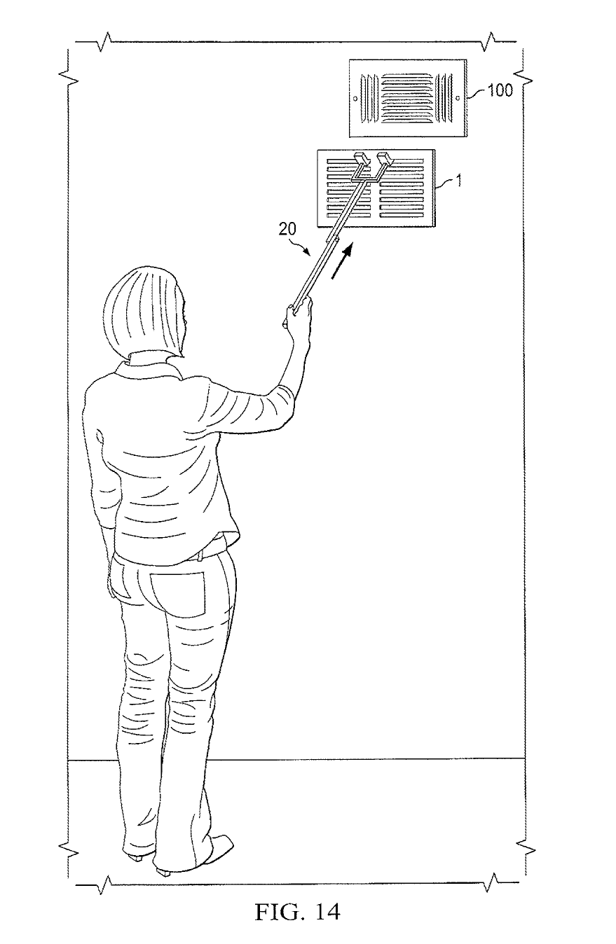

FIG. 14 is an environmental view that illustrates an example of the whole system of the invention. In this example, a user who is unable to reach the airflow register (100) may utilize the accessory aide (20) to help with attachment and detachment of the airflow register accessory (1) to and from the airflow register (100). The user will insert the holding extension supports (27) into the vent openings (10) so that holding extension support platforms (27a) extend through the vent openings (10) with the top edge of vent openings (10) resting thereon.

Holding extension support backings (27b) keep the airflow register accessory from sliding down the accessory aide (20). The outer side (12) of the accessory (1) may rest adjacent to the holding extension support backings (27b). The user may then extend out their arms, while holding the accessory aide (20), out towards the airflow register. The user will continue to extend out until the accessory (1), which is resting on the holding extension supports (27), attaches to the airflow register (100).

The user may also utilize the accessory aide (20) to help remove the airflow register accessory (1) from the airflow register (100). The holding extension supports (27) are inserted into the vent openings (10) as described above so that holding extension support platforms (27a) extend through the vent openings (10) with the top edge of vent openings (10) resting thereon. The user will then raise or lower the accessory aide (20) until the applied pressure of the holding extension supports (27) pushing against the airflow register (100) causes the fastening means (3) to release its grip and detach airflow register accessory (1) from the airflow register (100).

The accessory may be made in a plethora of decorative finishes thus allowing a user to easily change the décor in a room. Alternatively, the accessory may mimic the finish of the airflow register to which it is attached. Preferably, the accessory is made from a light-weight material such as a plastic or a metal.

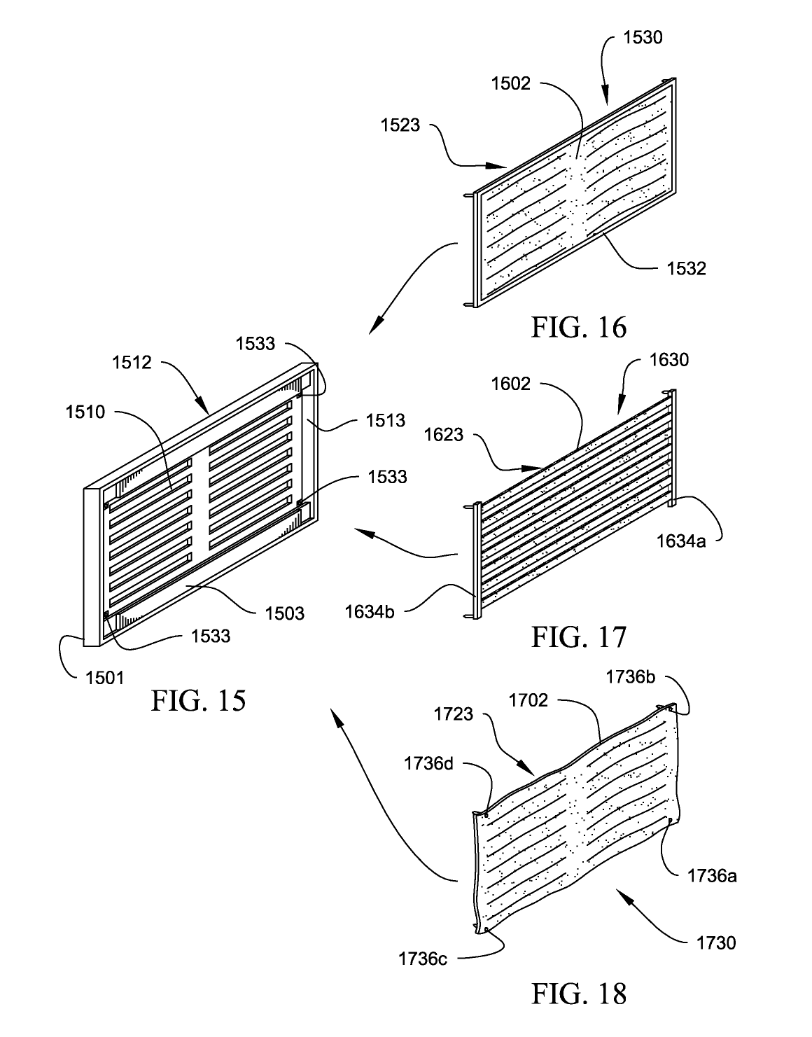

Now referring to FIG. 15, airflow register accessory (1501) has a top edge, a bottom edge, a left edge, a right edge, an outer surface (1512) and an inner surface (1513) and defines a plurality of vents (1510) for air to flow through the airflow register accessory (1501). The airflow register accessory (1501) further comprises a fastening mechanism (1503) secured to the inner surface (1513) of the accessory. The fastening mechanism (1503) is attached to the inner surface (1513) of the airflow register accessory (1501), and in turn may be used to secure the airflow register accessory (1501) to an airflow register. Preferably the fastening mechanism (1503) is secured to the top and bottom of the inner surface (1513) to avoid interference with screws, vent levers, and the like of a typical airflow register, but may be secured anywhere along the inner surface (1513) as to allow it to temporarily secure to any type of airflow register.

If the airflow register of interest is metal to which a magnet can adhere, the fastening mechanism can be one or more magnets. This allows for quick and easy attachment or detachment of the airflow register accessory (1501) from the airflow register of the room. Magnetic tape strips may also be used. If the accessory is made from non-magnetic material, it is convenient to use a magnetic tape strip that has an adhesive side for adhering to the inner surface of the accessory.

Other fastening mechanisms such as hook and loop fasteners may be employed (not illustrated). In such case, the complement of the hook or the loop is attached to the airflow register and the accessory secured thereto via the complementary fastener. Double-sided adhesive may also be used to attach the accessory to the airflow register. Less preferred, but possible, are fastening mechanism such as screws or bolts as they can secure the accessory to the airflow register, but make it more arduous to remove and replace the air accessory. If such are used, preferably they will extend through both the airflow register accessory and the airflow register and secure both to the wall, ceiling, or floor.

Alternatively, accessory (1501) may comprise attachment extensions that extend inward from the inner surface (1513). Fastening mechanisms (1503) would be secured to the attachment mechanisms. By providing the attachment extensions, the thickness of the accessory may be varied and allow the same fastening mechanisms to be used. The extensions may also be cost effective as it allows for thinner fastening mechanisms to be used.

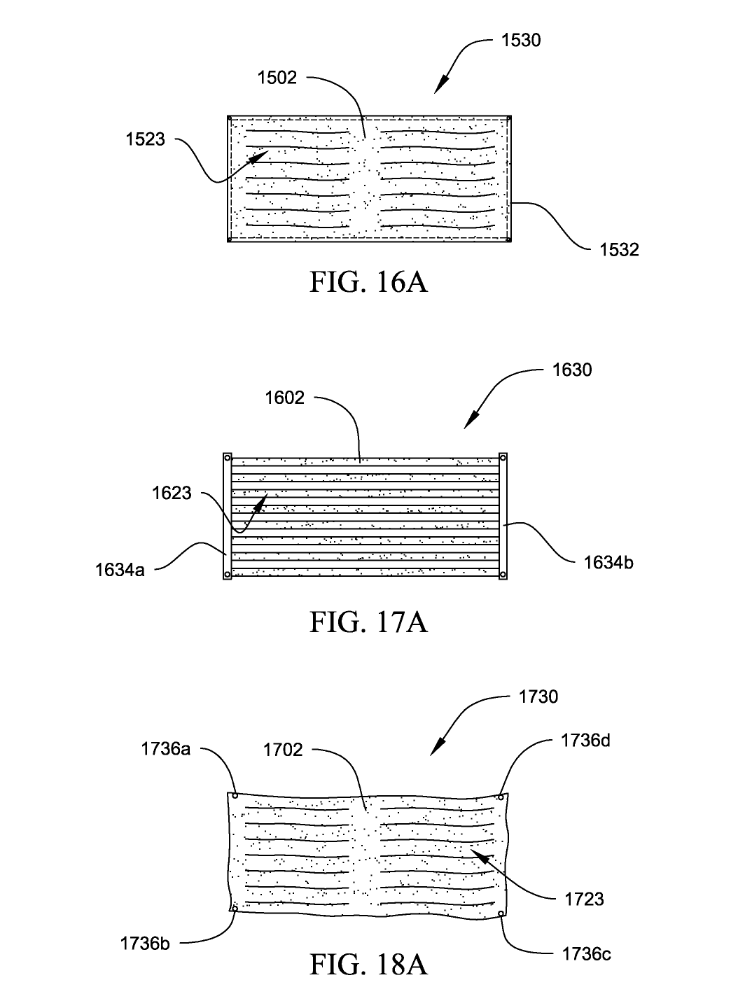

The accessory (1501) is further adapted to allow securement of a scenting material as shown in FIGS. 16 and 16A. FIGS. 16 and 16A illustrate a scented insert (1530) embodiment used for the scenting material. The scented insert (1530) comprises a scenting substrate (1502) and a substrate frame (1532) and has an accessory facing surface (1523) and a register facing surface. In this embodiment, the scenting substrate (1502) has a planar-like shape with outer edges, but other shapes may be used. The substrate frame (1532) is secured to the outer edges of the scenting substrate (1502) to form an outer border of the scented insert (1530). Scented insert (1530) has an accessory facing surface (1523) and an air register facing surface.

The airflow register accessory (1501) of the embodiment of FIG. 15 further has one or more insert connectors (1533) secured to the inner surface (1513) of the airflow register accessory (1501) which are adapted to engage scented insert (1530) and secure scented insert (1530) to the airflow register accessory (1501). The scented insert (1530) in FIGS. 16 and 16A is secured to the inner surface (1513) of accessory (1501) with the accessory facing surface (1523) of scented insert (1530) adjacent the vents (1510). Preferably, insert connectors (1533) engage the substrate frame (1532) of the scented insert (1530) and allow the substrate frame (1532) to “snap” into the insert connectors (1533). Other connector types may be clasps, clips, button snaps, removable adhesive or any type of connector that allows for securement of scented insert (1530) to the airflow register accessory (1501). Alternatively, if attachment extensions are used, the attachment extensions may also double as the insert connectors.

Preferably, the scenting substrate (1502) is a fabric sheet perforated or otherwise woven or spun so that it allows sufficient airflow therethrough (as illustrated in FIGS. 5 and 6). The fabric sheet has an air freshening agent loaded thereon, which is a scented oil or additive. The scented additive can include an essential oil or concentrated additive containing a concentrated fragrance or scent. When disposed on the fabric sheet, the scented additive is diluted with a water-based diluent up to 80% to 90% (or over) per weight of the scented additive. The diluent evaporates after application leaving the scented additive on the scented scenting substrate (1502). The air freshening agent must be able to adhere to the fabric but at least a portion thereof should be released when air flows through the fabric component. In the natural operation of the HVAC system, heated or cooled air will flow through the scenting material. Thus the air freshening agent may be chosen to release when air of any temperature flows through the scenting material at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting fabric so that the air freshening accessory can provide air freshening benefits for a pre-determined period of time.

Alternatively, a scented strip may be used instead of the fabric sheet. The scented strip would comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors. One or more slits which are preferably oriented in the same way as the vents may be made in the scented strip to allow air flow through the scented strip.

The scenting substrate (1502) may also be an adhesive substrate or a gel. After a pre-determined period of time, it is contemplated that the scented insert (1530) may be replaced.

The scented insert embodiment of FIGS. 16 and 16A may also comprise an air filter. Preferably, the scented insert (1530) comprises both the scenting substrate (1502) and the air filter with the air filter adjacent the scenting substrate (1502) on the accessory facing surface (1523) or the air register facing surface of the scented insert (1530). Alternatively, the air filter may be an individual structure secured to the airflow register accessory (1501). Any type of air filter may be used as long as it fits inside the airflow register accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

The accessory (1601) is further adapted to allow securement of a scenting material as shown in FIGS. 17 and 17A. FIGS. 17 and 17A illustrate a scented insert (1630) embodiment used for the scenting material. The scented insert (1630) comprises a scenting substrate (1602) and substrate braces (1634a and 1634b) and has an accessory facing surface (1623) and a register facing side. In this embodiment, the scenting substrate (1602) is one or more strips or bars that lie in a planar direction when in use, with each strip or bar having two ends. The scenting substrate (1602) can be rigid or non-rigid. Substrate brace (1634a) is secured to one end and substrate brace (1634b) is secured to the other end. Scented insert (1630) has an accessory facing surface (1623) and an air register facing surface.

The airflow register accessory (1501) of the embodiment of FIG. 15 further has one or more insert connectors (1533) secured to the inner surface (1513) of the airflow register accessory (1501) which are adapted to engage scented insert (1630) of FIGS. 17 and 17A and secure scented insert (1630) to the airflow register accessory (1501). The scented insert (1630) in FIGS. 17 and 17A is secured to the inner surface (1513) of accessory (1501) with the accessory facing surface (1623) of scented insert (1630) adjacent the vents (1510). Preferably, insert connectors (1533) engage the substrate braces (1634a and 1634b) of the scented insert (1630) and allow the substrate braces (1634a and 1634b) to “snap” into the insert connectors (1533). Other connector types may be clasps, clips, button snaps, removable adhesive or any type of connector that allows for securement of scented insert (1630) to the airflow register accessory (1501). Alternatively, if attachment extensions are used, the attachment extensions may also double as the insert connectors.

Preferably, the scenting substrate (1602) has an adhesive substrate or gel along the surface of or integrated into rigid bars. The adhesive substrate or gel would be impregnated with a fragrance. Preferably the scented surface has a covering that maintains the fragrance until use. The fragrance may be gel-based or oil-based. Preferably a fragrance that is long-lasting and safe to use indoors. One or more slits which are preferably oriented in the same way as the vents may be made in the scented strip to allow air flow through the scented strip.

Alternatively, the scenting substrate (1602) is one or more strips perforated or otherwise woven or spun so that it allows sufficient airflow therethrough (as illustrated in FIGS. 5 and 6). The strips have an air freshening agent loaded thereon, which is a scented oil or additive. The scented additive can include an essential oil or concentrated additive containing a concentrated fragrance or scent. When disposed on the strips, the scented additive is diluted with a water-based diluent up to 80% to 90% (or over) per weight of the scented additive. The diluent evaporates after application leaving the scented additive on the scenting substrate (1602). The air freshening agent must be able to adhere to the strips but at least a portion thereof should be released when air flows through the strips. In the natural operation of the HVAC system, heated or cooled air will flow through the scenting substrate.

Thus the air freshening agent may be chosen to release when air of any temperature flows through the scenting substrate at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting substrate so that the air freshening accessory can provide air freshening benefits for a pre-determined period of time.

After a pre-determined period of time, it is contemplated that the scented insert (1630) may be replaced.

The embodiment of FIGS. 17 and 17A may also comprise an air filter. Preferably, the scented insert (1630) comprises both the scenting substrate (1602) and the air filter with the air filter adjacent the scenting substrate (1602) on the accessory facing surface (1623) or the air register facing surface of the scented insert (1630). Alternatively, the air filter may be an individual structure secured to the airflow register accessory (1501). Any type of air filter may be used as long as it fits inside the airflow register accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

The accessory (1701) is further adapted to allow securement of a scenting material as shown in FIGS. 18 and 18A. FIGS. 18 and 18A illustrate a scented insert (1730) used for the scenting material. The scented insert (1730) comprises a scenting substrate (1702) and substrate connectors (1736a, 1736b, 1736c and 1736d). In this embodiment, the scenting substrate (1702) has a planar-like shape with outer edges, but other shapes may be used. The substrate connectors (1736a, 1736b, 1736c and 1736d) are secured to the outer edges of the scenting substrate (1702). Scented insert (1730) has an accessory facing surface (1723) and an air register facing surface. Although FIG. 17 illustrates four substrate connectors, more or less substrate connectors may be used for securing the scented insert to the air register accessory.

The airflow register accessory (1501) of the embodiment of FIG. 15 further has one or more insert connectors (1533) secured to the inner surface (1513) of the airflow register accessory (1501) which are adapted to engage the substrate connectors (1736a, 1736b, 1736c and 1736d) of scented insert (1730) of FIGS. 18 and 18A and secure scented insert (1730) to the airflow register accessory (1501). The scented insert (1730) in FIGS. 18 and 18A is secured to the inner surface (1513) of accessory (1501) with the accessory facing surface (1723) of scented insert (1730) adjacent the vents (1510). Insert connectors (1533) engage the substrate connectors (1736a, 1736b, 1736c and 1736d) of the scented insert (1730) and allow the substrate connectors (1736a, 1736b, 1736c and 1736d) to “snap” into the insert connectors (1533). Other connector types may be clasps, clips, button snaps, removable adhesive or any type of connector that allows for securement of scented insert (1730) to the airflow register accessory (1501). Alternatively, if attachment extensions are used, the attachment extensions may also double as the insert connectors.

Preferably, the scenting substrate (1702) is a fabric sheet perforated or otherwise woven or spun so that it allows sufficient airflow therethrough (as illustrated in FIGS. 5 and 6). The fabric sheet has an air freshening agent loaded thereon, which is a scented oil or additive. The scented additive can include an essential oil or concentrated additive containing a concentrated fragrance or scent. When disposed on the fabric sheet, the scented additive is diluted with a water-based diluent up to 80% to 90% (or over) per weight of the scented additive. The diluent evaporates after application leaving the scented additive on the scented scenting substrate (1702). The air freshening agent must be able to adhere to the fabric but at least a portion thereof should be released when air flows through the fabric component. In the natural operation of the HVAC system, heated or cooled air will flow through the scenting material. Thus the air freshening agent may be chosen to release when air of any temperature flows through the scenting material at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting fabric so that the air freshening accessory can provide air freshening benefits for a pre-determined period of time.

Alternatively, a scented strip may be used instead of the fabric sheet. The scented strip would comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors. One or more slits which are preferably oriented in the same way as the vents may be made in the scented strip to allow air flow through the scented strip.

The scenting substrate (1702) may also be an adhesive substrate or a gel. After a pre-determined period of time, it is contemplated that the scented insert (1730) may be replaced.

The embodiment of FIGS. 18 and 18A may also comprise an air filter. Preferably, the scented insert (1730) comprises both the scenting substrate (1702) and the air filter with the air filter adjacent the scenting substrate (1702) on the accessory facing surface (1723) or the air register facing surface of the scented insert (1730). Alternatively, the air filter may be an individual structure secured to the airflow register accessory (1501). Any type of air filter may be used as long as it fits inside the airflow register accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

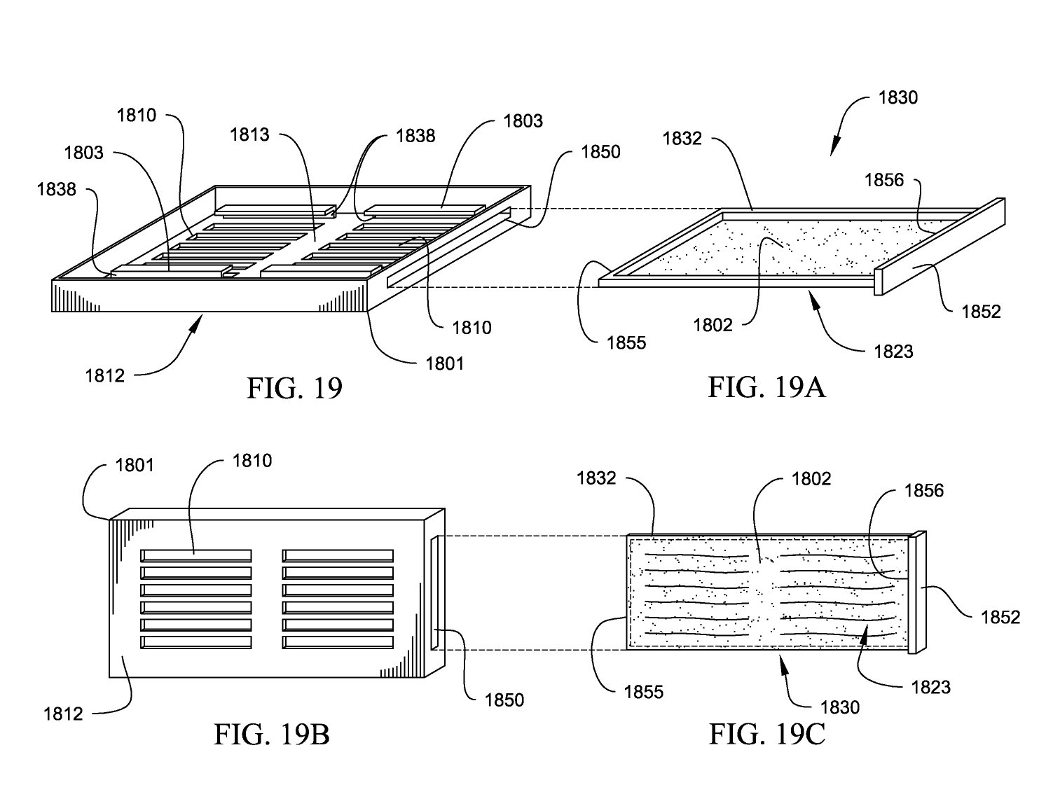

Now referring to FIGS. 19 and 19B, airflow register accessory (1801) has a top edge, a bottom edge, a left edge, a right edge, an outer surface (1812) and an inner surface (1813) and defines a plurality of vents (1810) for air to flow through the airflow register accessory (1801). The airflow register accessory (1801) further comprises a fastening mechanism (1803) secured to the inner surface (1813) of the accessory. The fastening mechanism (1803) is attached to the inner surface (1813) of the airflow register accessory (1801), and in turn may be used to secure the airflow register accessory (1801) to an airflow register. Preferably the fastening mechanism (1803) is secured to the top and bottom of the inner surface (1813) to avoid interference with screws, vent levers, and the like of a typical airflow register, but may be secured anywhere along the inner surface (1813) as to allow it to temporarily secure to any type of airflow register.

If the airflow register of interest is metal to which a magnet can adhere, the fastening mechanism can be one or more magnets. This allows for quick and easy attachment or detachment of the airflow register accessory (1801) from the airflow register of the room. Magnetic tape strips may also be used. If the accessory is made from non-magnetic material, it is convenient to use a magnetic tape strip that has an adhesive side for adhering to the inner surface of the accessory.

Other fastening mechanisms such as hook and loop fasteners may be employed (not illustrated). In such case, the complement of the hook or the loop is attached to the airflow register and the accessory secured thereto via the complementary fastener. Double-sided adhesive may also be used to attach the accessory to the airflow register. Less preferred, but possible, are fastening mechanism such as screws or bolts as they can secure the accessory to the airflow register, but make it more arduous to remove and replace the air accessory. If such are used, preferably they will extend through both the airflow register accessory and the airflow register and secure both to the wall, ceiling, or floor.

In the embodiment of FIGS. 19 and 19B, air register accessory (1801) also comprises attachment extensions (1838) that extend inward from the inner surface (1813) as shown in FIG. 19. The attachment extensions (1838) are adapted to receive an insert that slidably engages the attachment extensions (1838).

The fastening mechanisms (1803) may be secured to the attachment mechanisms (1838). By securing the fastening mechanisms (1803) to the attachment extensions (1838), the thickness of the accessory may be varied and allow the same fastening mechanisms to be used. The extensions may also be cost effective as it allows for thinner fastening mechanisms to be used.

The accessory (1801) is further adapted to allow securement of a scenting material as shown in FIGS. 19A and 19C. FIGS. 19A and 19C illustrate a scented insert (1830) used for the scenting material. The scented insert (1830) comprises a scenting substrate (1802) and a substrate frame (1832). In this embodiment, the scenting substrate (1802) has a planar-like shape with outer edges, but other shapes may be used. The substrate frame (1832) is secured to the outer edges of the scenting substrate (1802) to form an outer border of the scented insert (1830). Substrate frame (1832) has an inserting edge (1855) and a grasping edge (1856). Scented insert (1830) has an accessory facing surface (1823) and an air register facing surface.

The air register accessory (1801) of FIGS. 19 and 19B defines an insert slot (1850) along the left edge or right edge of the air register accessory (1801). The inserting edge (1855) of the scented insert (1830) of FIGS. 19A and 19C is passed though insert slot (1850). Scented insert (1830) continues to pass parallel to vents (1810) with accessory facing surface (1823) adjacent to the inner surface (1813) of the air register accessory (1801). As the scented insert (1830) slides adjacent to the inner surface (1813) of the air register accessory (1801), the substrate frame (1832) of scented insert (1830) slidably engages the attachment extensions (1838). The attachment extensions (1838) help the scented insert (1830) slide in that parallel motion and help maintain the vertical position on the scented insert (1830) relative to air register accessory (1801).

Scented insert (1830) continues to pass through insert slot (1850) until grasping edge (1856) is adjacent to the insert slot (1850). Preferably, the scented insert (1830) remains in that position until the need to be replaced. Alternatively, air register accessory (1801) may include insert connectors along the left or right edges or along attachment extensions (1838) that engage the substrate frame (1832) of the scented insert (1830) and allow the substrate frame (1832) to “snap” into the insert connectors (1833). Other connector types may be clasps, clips, button snaps, removable adhesive or any type of connector that allows for securement of scented insert (1832) to the airflow register accessory (1801).

Preferably, the scenting substrate (1802) is a fabric sheet perforated or otherwise woven or spun so that it allows sufficient airflow therethrough (as illustrated in FIGS. 5 and 6). The fabric sheet has an air freshening agent loaded thereon, which is a scented oil or additive. The scented additive can include an essential oil or concentrated additive containing a concentrated fragrance or scent. When disposed on the fabric sheet, the scented additive is diluted with a water-based diluent up to 80% to 90% (or over) per weight of the scented additive. The diluent evaporates after application leaving the scented additive on the scented scenting substrate (1802). The air freshening agent must be able to adhere to the fabric but at least a portion thereof should be released when air flows through the fabric component. In the natural operation of the HVAC system, heated or cooled air will flow through the scenting material. Thus the air freshening agent may be chosen to release when air of any temperature flows through the scenting material at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting fabric so that the air freshening accessory can provide air freshening benefits for a pre-determined period of time.

Alternatively, a scented strip may be used instead of the fabric sheet. The scented strip would comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors. One or more slits which are preferably oriented in the same way as the vents may be made in the scented strip to allow air flow through the scented strip.

The scenting substrate (1802) may also be an adhesive substrate or a gel. After a pre-determined period of time, it is contemplated that the scented insert (1830) may be replaced.

The embodiments shown in FIGS. 19, 19A, 19B and 19C may also comprise an air filter. Preferably, the scented insert (1830) of FIGS. 19A and 19C comprise both the scenting substrate (1802) and the air filter with the air filter adjacent the scenting substrate (1802) on the accessory facing surface (1823) or the air register facing surface of the scented insert (1830). Alternatively, the air filter may be an individual structure secured to the airflow register accessory (1801) of FIGS. 19 and 19B. Any type of air filter may be used as long as it fits inside the airflow register accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

Preferably, the airflow register accessory is secured to the airflow register in a manner of which any parts of the airflow register accessory or the scented insert do not interfere with levers, switches, or necessary components of the airflow register.

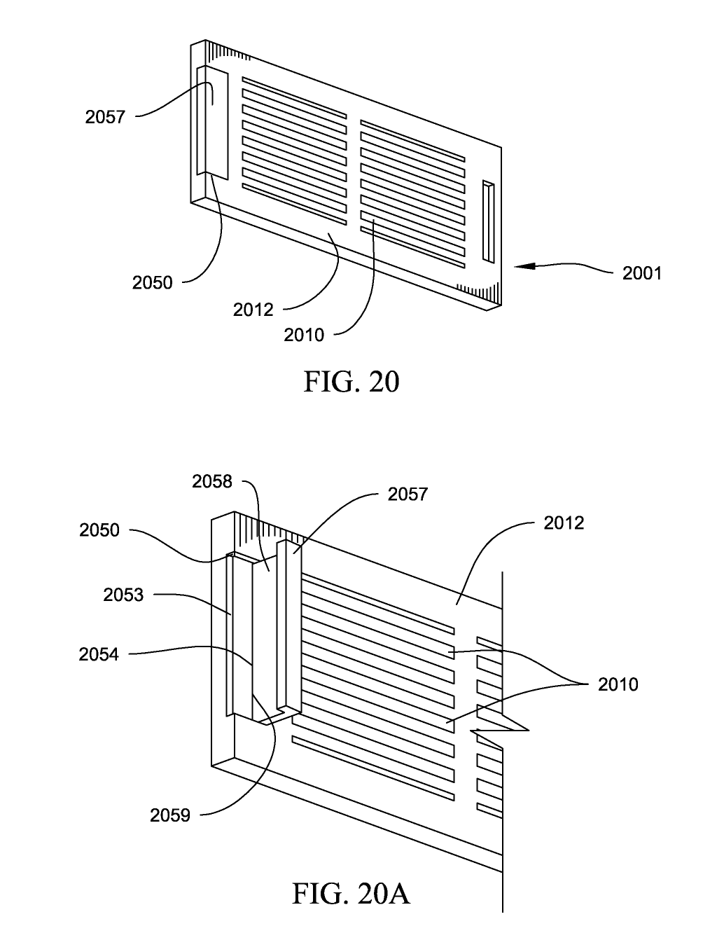

Now referring to FIG. 20 which illustrates an alternate embodiment of the area surrounding an insert slot. Airflow register accessory (2001) has a top edge, a bottom edge, a left edge, a right edge, an outer surface (2012) and an inner surface (not shown) and defines a plurality of vents (2010) for air to flow through the airflow register accessory (2001). The airflow register accessory (2001) further comprises a fastening mechanism (not shown) secured to the inner surface (2013) of the accessory. Air register accessory (2001) also comprises attachment extensions (not shown) that extend inward from the inner surface. The attachment extensions are adapted to receive an insert that slidably engages the attachment extensions.

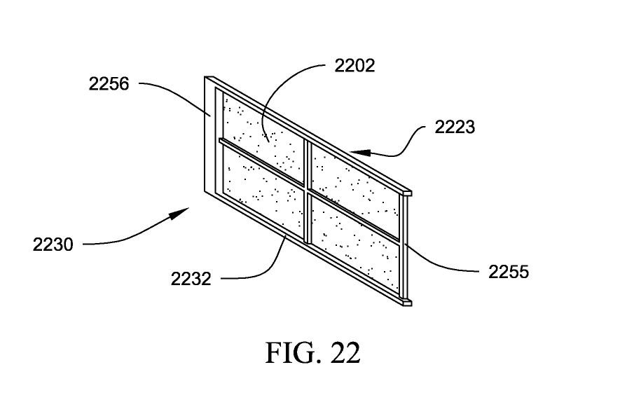

The accessory (2001) is further adapted to allow securement of a scenting material in the form of a scented insert as shown in FIGS. 19A, 19C and 22. The air register accessory (2001) defines an insert slot (2050) along the left edge or right edge of the air register accessory (2001) and extends centrally towards vents (2010). Insert slot (2050) has an accessory side edge (2053) and a vent side edge (2054). Airflow register accessory (2001) also comprises a slot cover (2057) having a vent side edge (2059) and an accessory side edge (2058). Vent side edge (2059) of slot cover (2057) is secured to vent side edge (2054) of insert slot (2050).

The secured vent side edges (2054 and 2059) act as a hinge-like structure the allows slot cover (2057) to change back and forth from a closed position, as shown in FIG. 20, to an open position, as shown in FIG. 20A. FIG. 20A is a zoomed in perspective view of FIG. 20 of the area surrounding insert slot (2050) showing slot cover (2057) in an open position. As slot cover (2057) is brought to a closed position, accessory side edge (2058) engages accessory side edge (2053). Preferably, insert slot (2050) and slot cover (2057) comprise connectors or engaging lips along their edges that hold slot cover (2057) in a closed position when engaged. The connectors or engaging lips release their hold when user applies force in an opening motion.

Alternatively, slot cover (2057) is removably attached to insert slot (2050) with the open position being the slot cover (2057) removed and the closed position being the slot cover (2057) fully engaged with or attached to the insert slot (2050).

The inserting edge of the scented insert of FIGS. 19A, 19C and 22 is passed though insert slot (2050). The scented insert continues to pass parallel to vents (2010) with an accessory facing surface adjacent to the inner surface of the air register accessory (2001). As the scented insert slides adjacent to the inner surface of the air register accessory (2001), the scented insert slidably engages the attachment extensions. The attachment extensions help the scented insert (2030) slide in that parallel motion and help maintain the vertical position on the scented insert (2030) relative to air register accessory (2001).

The scented insert continues to pass through insert slot (2050) until the grasping edge is adjacent to the insert slot (2050) on the inner side of the accessory as to allow slot cover (2057) to completely rest in a closed position. Slot cover (2057) is moved into a closed position thereby entrapping the scented insert (2057) along the inner side of the accessory. Preferably, the scented insert remains in that position until the need to be replaced.

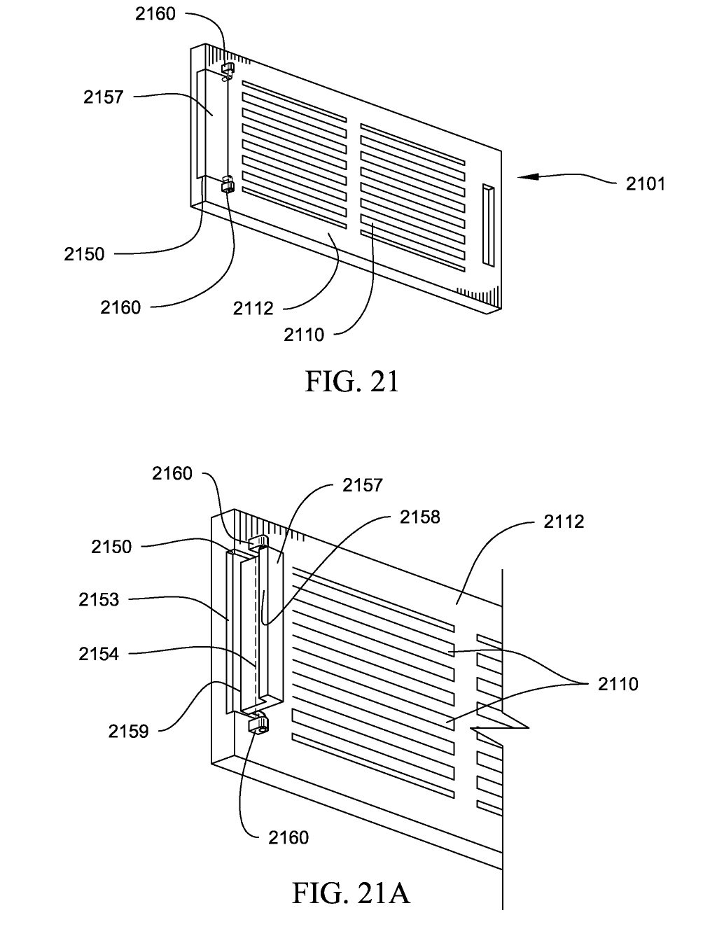

Now referring to FIG. 21 which illustrates an alternate embodiment of the area surrounding an insert slot. Airflow register accessory (2101) has a top edge, a bottom edge, a left edge, a right edge, an outer surface (2112) and an inner surface (not shown) and defines a plurality of vents (2110) for air to flow through the airflow register accessory (2101). The airflow register accessory (2101) further comprises a fastening mechanism (not shown) secured to the inner surface (2113) of the accessory. Air register accessory (2101) also comprises attachment extensions (not shown) that extend inward from the inner surface. The attachment extensions are adapted to receive an insert that slidably engages the attachment extensions.

The accessory (2101) is further adapted to allow securement of a scenting material in the form of a scented insert as shown in FIGS. 19A, 19C and 22. The air register accessory (2101) defines an insert slot (2150) along the left edge or right edge of the air register accessory (2101) and extends centrally towards vents (2110). Insert slot (2150) has an accessory side edge (2153) and a vent side edge (2154). Airflow register accessory (2101) comprises a slot cover (2157) having a vent side edge (2159) and an accessory side edge (2158). The outer surface (2112) of airflow register accessory (2101) comprises hinges (2160) secured approximate the vent side edge (2154) of insert slot (2150). Vent side edge (2159) of slot cover (2157) is coupled to hinges (2160) and lies adjacent to vent side edge (2154) of insert slot (2150). Alternatively, hinges (2160) are secured to the vent side edge (2159) of slot cover (2157) and coupled to the airflow register accessory (2101).

The hinges (2160) allow slot cover (2157) to change back and forth from a closed position, as shown in FIG. 21, to an open position, as shown in FIG. 21A. FIG. 21A is a zoomed in perspective view of FIG. 21 of the area surrounding insert slot (2150) showing slot cover (2157) in an open position. As slot cover (2157) is brought to a closed position, accessory side edge (2158) engages accessory side edge (2153). Preferably, insert slot (2150) and slot cover (2157) comprise connectors or engaging lips along their edges that hold slot cover (2157) in a closed position when engaged. The connectors or engaging lips release their hold when user applies force in an opening motion.

The inserting edge of the scented insert of FIGS. 19A, 19C and 22 is passed though insert slot (2150). The scented insert continues to pass parallel to vents (2110) with an accessory facing surface adjacent to the inner surface of the air register accessory (2101). As the scented insert slides adjacent to the inner surface of the air register accessory (2101), the scented insert slidably engages the attachment extensions. The attachment extensions help the scented insert (2130) slide in that parallel motion and help maintain the vertical position on the scented insert (2130) relative to air register accessory (2101).

The scented insert continues to pass through insert slot (2150) until the grasping edge is adjacent to the insert slot (2150) on the inner side of the accessory as to allow slot cover (2157) to completely rest in a closed position. Slot cover (2157) is moved into a closed position thereby entrapping the scented insert (2157) along the inner side of the accessory. Preferably, the scented insert remains in that position until the need to be replaced.

Now referring to FIG. 22, illustrating an embodiment of a scented insert. The scented insert (2230) comprises a scenting substrate (2202) and a substrate frame (2232). In this embodiment, the scenting substrate (2202) has a planar-like shape with outer edges, but other shapes may be used. The substrate frame (2232) is secured to the outer edges of the scenting substrate (2202) to form an outer border of the scented insert (2230). Substrate frame (2232) has an inserting edge (2255) and a grasping edge (2256). Scented insert (2230) has an accessory facing surface (2223) and an air register facing surface.

The air register accessory shown in FIGS. 19, 19B, 20 and 21 defines an insert slot along the left edge or right edge of the air register accessory and attachment extensions that extend inward from an inner surface. The attachment extensions are adapted to receive the scented insert that slidably engages the attachment extensions. The inserting edge (2255) of the scented insert (2230) of FIG. 22 is passed though the insert slot. Scented insert (2230) continues to pass parallel to vents of the air register accessory with accessory facing surface (2223) adjacent to the inner surface of the air register accessory. As the scented insert (2230) slides adjacent to the inner surface of the air register accessory, the substrate frame (2232) of scented insert (2230) slidably engages the attachment extensions. The attachment extensions help the scented insert (2230) slide in that parallel motion and help maintain the vertical position on the scented insert (2230) relative to the air register accessory.

Scented insert (2230) continues to pass through the insert slot until grasping edge (2256) is adjacent to the insert slot. Preferably, the scented insert (2230) remains in that position until the need to be replaced. Alternatively, the air register accessory may include insert connectors along the left or right edges or along the attachment extensions that engage the substrate frame (2232) of the scented insert (2230) and allow the substrate frame (2232) to “snap” into the insert connectors. Other connector types may be clasps, clips, button snaps, removable adhesive or any type of connector that allows for securement of scented insert (2232) to the airflow register accessory.

Preferably, the scenting substrate (2202) is a fabric sheet perforated or otherwise woven or spun so that it allows sufficient airflow therethrough. The fabric sheet has an air freshening agent loaded thereon, which is a scented oil or additive. The scented additive can include an essential oil or concentrated additive containing a concentrated fragrance or scent. When disposed on the fabric sheet, the scented additive is diluted with a water-based diluent up to 80% to 90% (or over) per weight of the scented additive. The diluent evaporates after application leaving the scented additive on the scented scenting substrate (2202). The air freshening agent must be able to adhere to the fabric but at least a portion thereof should be released when air flows through the fabric component. In the natural operation of the HVAC system, heated or cooled air will flow through the scenting material. Thus the air freshening agent may be chosen to release when air of any temperature flows through the scenting material at a pre-determined rate, when air having a temperature above a certain threshold (heater in operation) flows through at a pre-determined rate, or when air having a temperature below a certain threshold flows through (air conditioner in operation) flows through at a pre-determined rate. Sufficient air freshening agent should continue to adhere to the scenting fabric so that the air freshening accessory can provide air freshening benefits for a pre-determined period of time.

Alternatively, a scented strip may be used instead of the fabric sheet. The scented strip would comprise a scented surface that is impregnated with a fragrance. Preferably the scented surface has a covering that maintains the fragrance. The fragrance would be activated when the covering is removed. For example, the covering may me a plastic coating or sheet that peels off. The fragrance may be gel-based or oil-based. Preferably a fragrance that is long-lasting and safe to use indoors. One or more slits which are preferably oriented in the same way as the vents may be made in the scented strip to allow air flow through the scented strip.

The scenting substrate (2202) may also be an adhesive substrate or a gel. After a pre-determined period of time, it is contemplated that the scented insert (2230) may be replaced.

The scented insert embodiment of FIG. 22 may also comprise an air filter. Preferably, the scented insert (2230) has the air filter adjacent the scenting substrate (2202) on the accessory facing surface (2223) or the air register facing surface of the scented insert (2230). Alternatively, the scented insert may have an air filter in place of the scenting substrate. Any type of air filter may be used as long as it fits inside the airflow register accessory and doesn't impede the attachment of the accessory to the air register. Some examples of air filters are fiberglass filters, HEPA filters or washable air filters. Some filters like polyester and pleated filters may not be suitable as they typically have a higher resistance to air flow.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention.

Having described the invention, we claim:

1. An airflow accessory, comprising:
an accessory body structure having a top edge, a bottom edge, a left edge, a right edge, an outer surface, an inner surface, and ventilation apertures that allow for air flow to be channeled therethrough, said accessory body structure capable of being placed on an exterior surface of an air register vent;
a fastening mechanism that couples said accessory body structure to said air register vent, said inner surface of said accessory body structure being contiguously situated next to said exterior surface of said air register vent; and,
a scented insert body with an accessory facing surface and an air register facing surface coupled proximate to said ventilation apertures and inner surface of the accessory body structure, said scented insert having a scenting substrate impregnated with a fragrance, said fragrance being emitted into the air flow channeled through the air register vent and apertures in the accessory body structure.

2. The airflow accessory of claim 1, further comprising:
an insert slot on accessory body structure, said accessory body structure having one or more attachment extensions that extend inward from the inner surface of the accessory body structure, said scented insert body slidably engaging the attachment extensions and coupled proximate to said ventilation apertures and inner surface of the accessory body structure.

3. The airflow accessory of claim 2, further comprising:
a slot cover on the accessory body structure having an accessory side edge positioned proximate to accessory body structure when slot cover is in a closed position.

4. The airflow accessory of claim 3, wherein a vent side edge of said slot cover attaches to the accessory body structure.

5. The airflow accessory of claim 3, further comprising:
one or more hinges proximate to a vent side edge of said insert slot, said hinges couple slot cover to the accessory body structure.

6. The airflow accessory of claim 1, wherein said fastening mechanism is a magnet, a nut and bolt assembly or a locking tab mechanism.

7. The airflow accessory of claim 1, further comprising:
an insert connector secured to the inner surface of the accessory body structure and adapted to receive and secure said scented insert to said accessory body structure.

8. The airflow accessory of claim 7, wherein said scented insert is coupled by pressure engagement with said insert connector.

9. An airflow accessory, comprising:
an accessory body structure having an outer surface, an inner surface, and ventilation apertures that allow for air flow to be channeled therethrough, said accessory body structure capable of being placed on an exterior surface of an air register vent and said accessory body is configured to be of a similar shape as the shape of the air register vent;
a fastening mechanism that couples said accessory body structure to said air register vent, said inner surface of said accessory body structure being contiguously situated next to said exterior surface of said air register vent; and,
a scented insert body with an accessory facing surface and an air register facing surface coupled proximate to said ventilation apertures on the accessory body structure, said scented insert having a scenting substrate impregnated with a fragrance, said fragrance being emitted into the air flow channeled through the air register vent and apertures in the accessory body structure.

10. The airflow accessory of claim 9, further comprising:
an insert slot on the accessory body structure, said accessory body structure having one or more attachment extensions that extend inward from the inner surface of the accessory body structure, said scented insert body slidably engaging the attachment extensions and coupled proximate to said ventilation apertures and inner surface of the accessory body structure.

11. The airflow accessory of claim 10, further comprising:
a slot cover on the accessory body structure having an accessory side edge positioned proximate to accessory body structure when slot cover is in a closed position.

12. The airflow accessory of claim 11, wherein a vent side edge of said slot cover attaches to the accessory body structure.

13. The airflow accessory of claim 11, further comprising:
one or more hinges proximate to a vent side edge of said insert slot, said hinges couple slot cover to the accessory body structure.

14. The airflow accessory of claim 9, wherein said fastening mechanism is a magnet, a nut and bolt assembly or a locking tab mechanism.

15. The airflow accessory of claim 9, further comprising:
insert connectors secured to the inner surface of the accessory body structure and adapted to receive and secure said scented insert to said accessory body structure.

16. The airflow accessory of claim 15, wherein said scented insert pressure snaps into said insert connectors.

17. A method of providing fragrance to airflow channeled through an air register vent, comprising the steps of:
providing an accessory body structure having an outer surface, an inner surface, and ventilation apertures that allow for air flow to be channeled therethrough,
placing said accessory body structure on an exterior surface of an air register vent, said accessory body is configured to be of a similar shape as the shape of the air register vent;
fastening said accessory body structure to said air register vent, said inner surface of said accessory body structure being contiguously situated next to said exterior surface of said air register vent;
placing a scented insert body having an accessory facing surface and an air register facing surface proximate to said ventilation apertures on the accessory body structure, said scented insert having a scenting substrate impregnated with a fragrance, said fragrance; and,
allowing said fragrance to be emitted into the air flow channeled through the air register vent and apertures in the accessory body structure.

18. The method of claim 17, further comprising the steps of:
providing said accessory body structure with an insert slot on the accessory body structure;
providing said accessory body structure with attachment extensions that extend inward from the inner surface of the accessory body structure; and
inserting said scented insert body through the insert slot whereby the scented insert slidably engages the attachment extensions until fully coupled proximate said ventilation apertures and inner surface of the accessory body structure.

19. The method of claim 18, further comprising the steps of:
providing a slot cover on the accessory body structure; and
positioning an accessory side edge of said slot cover proximate to accessory body structure when said slot cover is in a closed postion.

20. The method of claim 19, comprising the step of:
attaching a vent side edge of said slot cover to the accessory body structure.

21. The method of claim 19, comprising the steps of:
providing one or more hinges on the accessory body structure proximate to a vent side edge of said insert slot; and
coupling said slot cover to said hinges.

22. The method of claim 17, wherein said fastening mechanism is a magnet, a nut and bolt assembly or a locking tab mechanism.

23. The method of claim 17, further comprising the step of:
securing insert connectors to the inner surface of the accessory body structure, said insert connectors adapted to receive and secure said scented insert to said accessory body structure.

24. The method of claim 23, wherein said scented insert pressure snaps into said insert connectors.

25. The method of claim 17, where said fastening is performed using a connector, hook, or elastic strap.
